(19) United States
(12) Patent Application Publication
(10) Pub. No.: US 2022/0133691 A1
(43) Pub. Date: May 5, 2022

(54) LASOFOXIFENE TREATMENT OF VVA AND OSTEOPOROSIS IN SURVIVORS OF BREAST CANCER AND OTHER MALIGNANCIES

(71) Applicant: Sermonix Pharmaceuticals, Inc., Columbus, OH (US)

(72) Inventor: David J. PORTMAN, Columbus, OH (US)

(21) Appl. No.: 17/468,115

(22) Filed: Sep. 7, 2021

Related U.S. Application Data
(63) Continuation of application No. 16/341,027, filed on Apr. 10, 2019, now abandoned.
(60) Provisional application No. 62/406,862, filed on Oct. 11, 2016.

(51) Int. Cl. A61K 31/40; A61K 47/68; A61K 39/395; A61K 9/00; A61K 31/683; A61K 38/23; A61K 45/06; C07K 16/28; C07K 16/32; A61P 35/04

(52) U.S. Cl. CPC A61K 31/40; C07K 2317/24; A61K 39/3955; A61K 9/0034; A61K 31/683; A61K 38/23; A61K 45/06; C07K 16/2875; C07K 16/32; A61K 9/0053; A61K 9/0036; A61P 35/04; A61K 39/39558; C07K 16/2818; A61K 47/6855; C07K 2317/21; A61K 47/6803

(57) ABSTRACT

The disclosure provides methods for treating vulvovaginal atrophy (VVA) and osteoporosis in breast cancer survivors and survivors of other malignancies with an effective amount of lasofoxifene, a pharmaceutically acceptable salt thereof, or a prodrug thereof.

### 1. CROSS-REFERENCE TO RELATED APPLICATIONS

This application is a continuation of co-pending U.S. application Ser. No. 16/341,027, filed Apr. 10, 2019, which is a National Stage of International Application No. PCT/US2017/055971, filed Oct. 10, 2017, which claims the benefit of U.S. Provisional Application Ser. No. 62/406,862, filed Oct. 11, 2016, each of which is incorporated in its entirety by reference.

### 2. BACKGROUND OF THE INVENTION

A variety of treatments are currently available for treating breast cancer, such as surgery, radiation therapy, chemotherapy, hormone therapy, and targeted therapy. More are being developed, with nearly 2000 open clinical trials currently listed in the clinicaltrials.gov database. The survival rates of breast cancer patients have improved in recent years due to advances in both diagnosis and treatment. Caring for breast cancer survivors is becoming increasingly important as the number of women who have previously been diagnosed with breast cancer continues to rise.

Treatments of breast cancer can cause serious side effects in breast cancer survivors. Some breast cancer treatments, such as chemotherapy, hormonal therapy, and ovarian suppression, can contribute to osteoporosis and bone loss by increased osteoclastic activity and net loss of bone mineral. These treatments can also lead to vulvovaginal atrophy (VVA).

There thus remains a need to develop therapeutic strategies that effectively manage the risks of osteoporosis and VVA in breast cancer survivors, as well as in survivors of other malignancies.

### 3. SUMMARY OF THE INVENTION

In a first aspect, a method of treating vulvovaginal atrophy (VVA) in women who have previously been diagnosed with primary or metastatic breast cancer is presented. The method comprises selecting for treatment a patient with VVA who has previously been diagnosed with either i) estrogen receptor positive (ER+) breast cancer or ii) estrogen receptor negative (ER−) breast cancer; and administering to the selected patient lasofoxifene, a pharmaceutically acceptable salt thereof, or a prodrug thereof, in an amount effective to treat VVA.

In various embodiments, lasofoxifene is administered to the selected breast cancer patient as lasofoxifene tartrate. In various embodiments, lasofoxifene is administered by oral, intravenous, transdermal, vaginal topical, or vaginal ring administration. In certain embodiments, lasofoxifene is administered by oral administration. In some of these embodiments, lasofoxifene is administered at about 0.5 mg/day per os to about 10 mg/day per os. In certain embodiments, lasofoxifene is administered by vaginal topical administration. In certain embodiments, lasofoxifene is administered by vaginal ring administration. In various embodiments, lasofoxifene is administered once every day, once every two days, once every three days, once every four days, once every five days, once every six days, once every week, once every two weeks, once every three weeks, or once every month.

In some embodiments, the method further comprises treating the patient with at least one additional endocrine therapy. In certain embodiments, the additional endocrine therapy is treatment with a selective ER modulator (SERM) other than lasofoxifene. In certain embodiments, the additional endocrine therapy is treatment with a selective ER degrader (SERD). In certain embodiments, the additional endocrine therapy is treatment with an aromatase inhibitor.

In some embodiments, the method further comprises administering to the breast cancer patient an effective amount of cyclin-dependent kinase 4/6 (CDK4/6) inhibitor. In certain embodiments, CDK4/6 inhibitor is palbociclib, abemaciclib, or ribociclib. In some embodiments, the method further comprises administering to the patient an effective amount of mammalian target of rapamycin (mTOR) inhibitor, phosphoinositide 3-kinase (PI3K) inhibitor, or heat shock protein 90 (HSP90) inhibitor. In some embodiments, the method further comprises administering to the patient an effective amount of human epidermal growth factor receptor 2 (HER2) inhibitor. In certain embodiments, the HER2 inhibitor is trastuzumab (Herceptin®) or ado-trastuzumab emtansine (Kadcyla®). In some embodiments, the method further comprises administering to the patient an effective amount of a checkpoint inhibitor. In some of these embodiments, the checkpoint inhibitor is an antibody specific for programmed cell death protein 1 (PD-1), programmed death-ligand 1 (PD-L1), or cytotoxic T-lymphocyte-associated protein 4 (CTLA-4). In certain embodiments, the PD-1 antibody is pembrolizumab (Keytruda) or nivolumab)(Opdivo®. In certain embodiments, the CTLA-4 antibody is ipilimumab (Yervoy®). In some embodiments, the method further comprises administering to the patient an effective amount of cancer vaccine.

In some embodiments, the method comprises administering an amount of lasofoxifene sufficient to decrease vaginal pH, increase vaginal lubrication, and/or improve vaginal cell maturation index in women who are concurrently being treated with one or more drugs causing or predisposing to VVA. In some embodiments, the method comprises administering an amount of lasofoxifene sufficient to reduce one or more symptoms of sexual dysfunction in women who are concurrently being treated with one or more drugs causing or predisposing to sexual dysfunction. In some embodiments, the method comprises administering an amount of lasofoxifene sufficient to reduce one or more symptoms of hot flashes in women who are concurrently being treated with one or more drugs causing or predisposing to hot flashes. In some embodiments, the method comprises administering an amount of lasofoxifene sufficient to reduce recurrence of breast cancer, increase time to recurrence of breast cancer, reduce metastasis of breast cancer to bone, and/or increase duration of breast cancer progression-free survival. In some embodiments, the method comprises administering an amount of lasofoxifene sufficient to increase one or more quality of life measures selected from: joint ache, urogenital symptoms, bone loss, and bone fractures.

In certain embodiments, the patient's breast cancer is in remission.

In another aspect, a method of treating vulvovaginal atrophy (VVA) in women who have previously been diagnosed with a malignancy other than breast cancer is presented. The method comprises selecting for treatment a patient with VVA who has previously been diagnosed with either i) estrogen receptor positive (ER+) malignancy other than breast cancer or ii) estrogen receptor negative (ER−) malignancy other than breast cancer, and administering to the selected patient lasofoxifene, a pharmaceutically acceptable salt thereof, or a prodrug thereof, in an amount effective to treat VVA.

In various embodiments, lasofoxifene is administered to the selected patient with a malignancy other than breast cancer as lasofoxifene tartrate. In various embodiments, lasofoxifene is administered by oral, intravenous, transdermal, vaginal topical, or vaginal ring administration. In certain embodiments, lasofoxifene is administered by oral administration. In some of these embodiments, lasofoxifene is administered at about 0.5 mg/day per os to about 10 mg/day per os. In certain embodiments, lasofoxifene is administered by vaginal topical administration. In certain embodiments, lasofoxifene is administered by vaginal ring administration. In various embodiments, lasofoxifene is administered once every day, once every two days, once every three days, once every four days, once every five days, once every six days, once every week, once every two weeks, once every three weeks, or once every month.

In some embodiments, the method further comprises treating the patient with at least one additional endocrine therapy. In certain embodiments, the additional endocrine therapy is treatment with a selective ER modulator (SERM) other than lasofoxifene. In certain embodiments, the additional endocrine therapy is treatment with a selective ER degrader (SERD). In certain embodiments, the additional endocrine therapy is treatment with an aromatase inhibitor.

In some embodiments, the method further comprises administering to the ER⁺ metastatic breast cancer patient an effective amount of cyclin-dependent kinase 4/6 (CDK4/6) inhibitor. In certain embodiments, CDK4/6 inhibitor is palbociclib, abemaciclib, or ribociclib. In some embodiments, the method further comprises administering to the patient an effective amount of mammalian target of rapamycin (mTOR) inhibitor, phosphoinositide 3-kinase (PI3K) inhibitor, or heat shock protein 90 (HSP90) inhibitor. In some embodiments, the method further comprises administering to the patient an effective amount of human epidermal growth factor receptor 2 (HER2) inhibitor. In certain embodiments, the HER2 inhibitor is trastuzumab (Herceptin®) or ado-trastuzumab emtansine)(Kadcyla®). In some embodiments, the method further comprises administering to the patient an effective amount of a checkpoint inhibitor. In some of these embodiments, the checkpoint inhibitor is an antibody specific for programmed cell death protein 1 (PD-1), programmed death-ligand 1 (PD-L1), or cytotoxic T-lymphocyte-associated protein 4 (CTLA-4). In certain embodiments, the PD-1 antibody is pembrolizumab (Keytruda) or nivolumab)(Opdivo®). In certain embodiments, the CTLA-4 antibody is ipilimumab (Yervoy®). In some embodiments, the method further comprises administering to the patient an effective amount of cancer vaccine.

In some embodiments, the method comprises administering an amount of lasofoxifene sufficient to decrease vaginal pH, increase vaginal lubrication, and/or improve vaginal cell maturation index in women who are concurrently being treated with one or more drugs causing or predisposing to VVA. In some embodiments, the method comprises administering an amount of lasofoxifene sufficient to reduce one or more symptoms of sexual dysfunction in women who are concurrently being treated with one or more drugs causing or predisposing to sexual dysfunction. In some embodiments, the method comprises administering an amount of lasofoxifene sufficient to reduce one or more symptoms of hot flashes in women who are concurrently being treated with one or more drugs causing or predisposing to hot flashes. In some embodiments, the method comprises administering an amount of lasofoxifene sufficient to reduce cancer recurrence, increase time to cancer recurrence, reduce metastasis of cancer to bone, and/or increase duration of cancer progression-free survival. In some embodiments, the method comprises administering an amount of lasofoxifene sufficient to increase one or more quality of life measures selected from: joint ache, urogenital symptoms, bone loss, and bone fractures.

In certain embodiments, the patient's malignancy is in remission.

In another aspect, a method of treating osteoporosis in women who have previously been diagnosed with primary or metastatic breast cancer is presented. The method comprises selecting for treatment a patient with osteoporosis who has previously been diagnosed with either i) estrogen receptor positive (ER+) breast cancer or ii) estrogen receptor negative (ER−) breast cancer, and administering to the selected patient lasofoxifene, a pharmaceutically acceptable salt thereof, or a prodrug thereof, in an amount effective to treat osteoporosis.

In various embodiments, lasofoxifene is administered to the selected breast cancer patient as lasofoxifene tartrate. In various embodiments, lasofoxifene is administered by oral, intravenous, transdermal, vaginal topical, or vaginal ring administration. In certain embodiments, lasofoxifene is administered by oral administration. In some of these embodiments, lasofoxifene is administered at about 0.5 mg/day per os to about 10 mg/day per os. In various embodiments, lasofoxifene is administered once every day, once every two days, once every three days, once every four days, once every five days, once every six days, once every week, once every two weeks, once every three weeks, or once every month.

In some embodiments, the method further comprises treating the patient with at least one additional endocrine therapy. In certain embodiments, the additional endocrine therapy is treatment with a selective ER modulator (SERM) other than lasofoxifene. In certain embodiments, the additional endocrine therapy is treatment with a selective ER degrader (SERD). In certain embodiments, the additional endocrine therapy is treatment with an aromatase inhibitor.

In some embodiments, the method further comprises administering to the ER⁺ metastatic breast cancer patient an effective amount of cyclin-dependent kinase 4/6 (CDK4/6) inhibitor. In certain embodiments, CDK4/6 inhibitor is palbociclib, abemaciclib, or ribociclib. In some embodiments, the method further comprises administering to the patient an effective amount of mammalian target of rapamycin (mTOR) inhibitor, phosphoinositide 3-kinase (PI3K) inhibitor, or heat shock protein 90 (HSP90) inhibitor. In some embodiments, the method further comprises administering to the patient an effective amount of human epidermal growth factor receptor 2 (HER2) inhibitor. In certain embodiments, the HER2 inhibitor is trastuzumab (Herceptin®) or ado-trastuzumab emtansine (Kadcyla®). In some embodiments, the method further comprises administering to the patient an effective amount of a checkpoint inhibitor. In some of these embodiments, the checkpoint inhibitor is an antibody specific for programmed cell death protein 1 (PD-1), programmed death-ligand 1 (PD-L1), or cytotoxic T-lymphocyte-associated protein 4 (CTLA-4). In certain embodiments, the PD-1 antibody is pembrolizumab (Keytruda®) or nivolumab)(Opdivo®). In certain embodiments, the CTLA-4 antibody is ipilimumab (Yervoy®). In some embodiments, the method further comprises administering to the patient an effective amount of cancer vaccine.

In some embodiments, the method further comprises administering to the patient an effective amount of bisphosphonate. In various embodiments, the bisphosphonate is selected from etidronate)(Didronel®), clodronate (Bonefos®, Loron®), tiludronate)(Skelid®), pamidronate) (Aredia®), neridronate)(Nerixia®), olpadronate, alendronate)(Fosamax®), ibandronate (Boniva®), risedronate (Actonel®, Atelvia®), and zoledronate (Zometa®, Aclasta®). In some embodiments, the method further comprises administering to said patient an effective amount of receptor activator of nuclear factor kappa-B ligand (RANKL) inhibitor. In some of these embodiments, the RANKL inhibitor is denosumab (Prolia®, Xgeva®). In some embodiments, the method further comprises administering to the patient an effective amount of calcitonin (Miacalcin®), Fortical®).

In some embodiments, the method comprises administering an amount of lasofoxifene sufficient to prevent fractures and bone loss in women who are concurrently being treated with one or more drugs causing or predisposing to osteoporosis. In some embodiments, the method comprises administering an amount of lasofoxifene sufficient to reduce recurrence of breast cancer, increase time to recurrence of breast cancer, reduce metastasis of breast cancer to bone, and/or increase duration of breast cancer progression-free survival. In some embodiments, the method comprises administering an amount of lasofoxifene sufficient to increase one or more quality of life measures selected from: joint ache, urogenital symptoms, bone loss, and bone fractures.

In certain embodiments, the patient's breast cancer is in remission.

In another aspect, a method of treating osteoporosis in women who have previously been diagnosed with a malignancy other than breast cancer is presented. The method comprises selecting for treatment a patient with osteoporosis who has previously been diagnosed with either i) estrogen receptor positive (ER+) malignancy other than breast cancer or ii) estrogen receptor negative (ER−) malignancy other than breast cancer, and administering to the selected patient lasofoxifene, a pharmaceutically acceptable salt thereof, or a prodrug thereof, in an amount effective to treat osteoporosis.

In various embodiments, lasofoxifene is administered to the selected patient with a malignancy other than breast cancer as lasofoxifene tartrate. In various embodiments, lasofoxifene is administered by oral, intravenous, transdermal, vaginal topical, or vaginal ring administration. In certain embodiments, lasofoxifene is administered by oral administration. In some of these embodiments, lasofoxifene is administered at about 0.5 mg/day per os to about 10 mg/day per os. In various embodiments, lasofoxifene is administered once every day, once every two days, once every three days, once every four days, once every five days, once every six days, once every week, once every two weeks, once every three weeks, or once every month.

In some embodiments, the method further comprises treating the patient with at least one additional endocrine therapy. In certain embodiments, the additional endocrine therapy is treatment with a selective ER modulator (SERM) other than lasofoxifene. In certain embodiments, the additional endocrine therapy is treatment with a selective ER degrader (SERD). In certain embodiments, the additional endocrine therapy is treatment with an aromatase inhibitor.

In some embodiments, the method further comprises administering to the ER⁺ metastatic breast cancer patient an effective amount of cyclin-dependent kinase 4/6 (CDK4/6) inhibitor. In certain embodiments, CDK4/6 inhibitor is palbociclib, abemaciclib, or ribociclib. In some embodiments, the method further comprises administering to the patient an effective amount of mammalian target of rapamycin (mTOR) inhibitor, phosphoinositide 3-kinase (PI3K) inhibitor, or heat shock protein 90 (HSP90) inhibitor. In some embodiments, the method further comprises administering to the patient an effective amount of human epidermal growth factor receptor 2 (HER2) inhibitor. In certain embodiments, the HER2 inhibitor is trastuzumab (Herceptin®) or ado-trastuzumab emtansine (Kadcyla®). In some embodiments, the method further comprises administering to the patient an effective amount of a checkpoint inhibitor. In some of these embodiments, the checkpoint inhibitor is an antibody specific for programmed cell death protein 1 (PD-1), programmed death-ligand 1 (PD-L1), or cytotoxic T-lymphocyte-associated protein 4 (CTLA-4). In certain embodiments, the PD-1 antibody is pembrolizumab (Keytruda®) or nivolumab (Opdivo®). In certain embodiments, the CTLA-4 antibody is ipilimumab (Yervoy®). In some embodiments, the method further comprises administering to the patient an effective amount of cancer vaccine.

In some embodiments, the method further comprises administering to the patient an effective amount of bisphosphonate. In various embodiments, the bisphosphonate is selected from etidronate)(Didronel®), clodronate (Bonefos®, Loron®), tiludronate)(Skelid®), pamidronate (Aredia®), neridronate)(Nerixia®), olpadronate, alendronate (Fosamax®), ibandronate (Boniva®), risedronate (Actonel®, Atelvia®), and zoledronate (Zometa®, Aclasta®). In some embodiments, the method further comprises administering to said patient an effective amount of receptor activator of nuclear factor kappa-B ligand (RANKL) inhibitor. In some of these embodiments, the RANKL inhibitor is denosumab (Prolia®, Xgeva®). In some embodiments, the method further comprises administering to the patient an effective amount of calcitonin (Miacalcin®, Fortical®).

In some embodiments, the method comprises administering an amount of lasofoxifene sufficient to prevent fractures and bone loss in women who are concurrently being treated with one or more drugs causing or predisposing to osteoporosis. In some embodiments, the method comprises administering an amount of lasofoxifene sufficient to reduce recurrence of breast cancer, increase time to recurrence of breast cancer, reduce metastasis of breast cancer to bone, and/or increase duration of breast cancer progression-free survival. In some embodiments, the method comprises administering an amount of lasofoxifene sufficient to increase one or more quality of life measures selected from: joint ache, urogenital symptoms, bone loss, and bone fractures.

In certain embodiments, the patient's malignancy is in remission.

### 4. DETAILED DESCRIPTION

Various therapies used for treatment of breast cancer predispose the patient to osteoporosis, with potential for bone fracture. Various therapies used for treatment of breast cancer predispose the patient to vulvovaginal atrophy (VVA), an inflammation of the vagina due to thinning and shrinking of the tissues, as well as decreased lubrication. Ovarian suppression, radiation therapy, and chemotherapy can cause decreased ovarian functioning, and therefore lead to VVA. Hormonal therapy can contribute to VVA by decreasing estrogen level or decreasing estrogen signaling.

Lasofoxifene is a selective ER modulator (SERM). It has high binding affinity for the estrogen receptor and acts as a tissue-selective estrogen agonist or antagonist. In the double-blind, placebo-controlled, randomized Postmenopausal Evaluation and Risk-Reduction with Lasofoxifene (PEARL) trial, lasofoxifene was found to reduce the risk of osteoporosis. See Cummings el al., The New England Journal of Medicine 326(8): 686-696 (2010). In the PEARL trial, it was also found that lasofoxifene reduced the risk of breast cancer in post-menopausal women with osteoporosis. See LaCroix el al., Journal of the National Cancer Institute 102(22): 1706-1715 (2010). However, the effect of lasofoxifene on VVA and osteoporosis specifically in breast cancer survivors was not assessed.

### 4.1. Patient Selection

In a first aspect, disclosed herein are methods of treating vulvovaginal atrophy (VVA) and/or osteoporosis in women who have previously been diagnosed with breast cancer. The methods comprise selecting for treatment a patient who has been diagnosed with VVA and/or osteoporosis, and either i) estrogen receptor positive (ER+) cancer or ii) estrogen receptor negative (ER−) cancer. The selected patient is treated with an effective amount of lasofoxifene, a pharmaceutically acceptable salt thereof, or a prodrug thereof.

### 4.1.1. Breast Cancer Survivors with Vulvovaginal Atrophy (VVA)

In a first series of embodiments, the patient has VVA and has previously been diagnosed with breast cancer.

In typical embodiments, the patient has been diagnosed with Vulvovaginal Atrophy (VVA). In certain embodiments, the patient has been diagnosed with VVA by genital symptoms. In certain embodiments, the patient has been diagnosed with VVA by urinary symptoms. In certain embodiments, the patient's VVA is characterized by vaginal dryness, irritation, and soreness. In certain embodiments, the patient has a vaginal pH of 4.6 or higher. In certain embodiments, the patient has one or more symptoms of sexual dysfunction. In certain embodiments, the patient has one or more symptoms of hot flashes.

In various embodiments, the patient is predisposed to Vulvovaginal Atrophy (VVA). In some embodiments, the patient is postmenopausal. In some embodiments, the patient has decreased level of circulating estrogen. In some embodiments, the patient has previously been diagnosed with an immunological disorder.

In some embodiments, the VVA patient has previously been diagnosed with primary breast cancer. In some embodiments, the VVA patient has previously been diagnosed with metastatic breast cancer.

In some embodiments, the VVA patient has previously been diagnosed with estrogen receptor positive (ER+) breast cancer. In some other embodiments, the VVA patient has previously been diagnosed with estrogen receptor negative (ER−) breast cancer. In various embodiments, the patient has been diagnosed with ER+ or ER− breast cancer by immunohistochemistry (IHC) performed on a sample of the patient's cancer.

In some embodiments, the patient's breast cancer has previously been treated with surgery. In some embodiments, the patient has previously been treated with radiation therapy. In some embodiments, the patient has previously been treated with chemotherapy. In some embodiments, the patient has previously been treated with targeted therapy.

In some embodiments, the patient has previously been treated with hormone therapy.

In some embodiments, the hormone therapy that the patient has previously been treated with is ovarian suppression. In certain embodiments, ovarian suppression is achieved by oophorectomy. In certain embodiments, ovarian suppression is achieved by administration of a GnRH antagonist.

In some embodiments, the hormone therapy that the patient has previously been treated with is an aromatase inhibitor. In some embodiments, the aromatase inhibitor is selected from exemestane (Aromasin®), letrozole (Femara®), and anastrozole (Arimidex®).

In some embodiments, the hormone therapy that the patient has previously been treated with is a selective ER modulator (SERM) other than lasofoxifene. In some embodiments, the selective ER modulator is selected from tamoxifen, raloxifene, bazedoxifene, toremifene, and ospemifene. In certain embodiments, the selective ER modulator is tamoxifen.

In some embodiments, the hormone therapy that the patient has previously been treated with is a selective ER degrader (SERD). In various embodiments, the selective ER degrader binds to the estrogen receptor and leads to the proteasomal degradation of the receptor. In some embodiments, the selective ER degrader is selected from fulvestrant, RAD1901, ARN-810 (GDC-0810), and AZD9496. In certain embodiments, the selective ER degrader is fulvestrant.

In certain embodiments, the VVA patient who has previously been treated with hormone therapy is continuing to receive hormone therapy.

In some embodiments, the VVA patient's breast cancer is in remission. In some of these embodiments, the patient's breast cancer is in partial remission. In some other of these embodiments, the patient's breast cancer is in complete remission.

### 4.1.2. Other Cancer Survivors with Vulvovaginal Atrophy (VVA)

In another series of embodiments, the VVA patient has previously been diagnosed with a malignancy other than breast cancer. In some of these embodiments, the VVA patient has previously been diagnosed with ovarian cancer. In some other of these embodiments, the VVA patient has previously been diagnosed with lung cancer.

In some embodiments, the VVA patient has previously been diagnosed with estrogen receptor positive (ER+) malignancy other than breast cancer. In some other embodiments, the VVA patient has previously been diagnosed with estrogen receptor negative (ER−) malignancy other than breast cancer. In various embodiments, the patient has been diagnosed with ER+ or ER- malignancy other than breast cancer by immunohistochemistry (IHC) performed on a sample of the patient's cancer.

In some embodiments, the patient's cancer has previously been treated with surgery. In some embodiments, the patient has previously been treated with radiation therapy. In some embodiments, the patient has previously been treated with chemotherapy. In some embodiments, the patient has previously been treated targeted therapy.

In some embodiments, the patient has previously been treated with hormone therapy.

In some embodiments, the hormone therapy that the patient has previously been treated with is ovarian suppression. In certain embodiments, ovarian suppression is achieved by oophorectomy. In certain embodiments, ovarian suppression is achieved by administration of a GnRH antagonist.

In some embodiments, the hormone therapy that the patient has previously been treated with is an aromatase inhibitor. In some embodiments, the aromatase inhibitor is selected from exemestane (Aromasin®), letrozole (Femara®), and anastrozole (Arimidex®).

In some embodiments, the hormone therapy that the patient has previously been treated with is a selective ER modulator (SERM) other than lasofoxifene. In some embodiments, the selective ER modulator is selected from tamoxifen, raloxifene, bazedoxifene, toremifene, and ospemifene. In certain embodiments, the selective ER modulator is tamoxifen.

In some embodiments, the hormone therapy that the patient has previously been treated with is a selective ER degrader (SERD). In various embodiments, the selective ER degrader binds to the estrogen receptor and leads to the proteasomal degradation of the receptor. In some embodiments, the selective ER degrader is selected from fulvestrant, RAD1901, ARN-810 (GDC-0810), and AZD9496. In certain embodiments, the selective ER degrader is fulvestrant.

In some embodiments, the VVA patient's malignancy is in remission. In some of these embodiments, the patient's malignancy is in partial remission. In some other of these embodiments, the patient's malignancy is in complete remission.

### 4.1.3. Breast Cancer Survivors with Osteoporosis

In various embodiments, the patient has been diagnosed with osteoporosis and has previously been diagnosed with breast cancer.

In some embodiments, the patient has been diagnosed with osteoporosis by a bone mineral density (BMD) test. In some of these embodiments the BMD test is a dual-energy x-ray absorptiometry (DEXA) scan. In some embodiments, the patient's osteoporosis is characterized by low bone mass and structural deterioration of bone tissue. In some embodiments, the patient's osteoporosis can lead to bone fracture.

In various embodiments, the patient is predisposed to osteoporosis. In some embodiments, the patient is postmenopausal. In some embodiments, the patient has decreased level of circulating estrogen. In some embodiments, the patient has previously been diagnosed with a chronic disease and has previously used medications that can impair calcium absorption.

In some of these embodiments, the osteoporosis patient has previously been diagnosed with primary breast cancer. In some embodiments, the osteoporosis patient has previously been diagnosed with metastatic breast cancer.

In some embodiments, the osteoporosis patient has previously been diagnosed with estrogen receptor positive (ER+) breast cancer. In some other embodiments, the osteoporosis patient has previously been diagnosed with estrogen receptor negative (ER−) breast cancer. In various embodiments, the patient has been diagnosed with ER+ or ER− breast cancer by immunohistochemistry (IHC) performed on a sample of the patient's cancer.

In some embodiments, the patient's cancer has previously been treated with surgery. In some embodiments, the patient has previously been treated with radiation therapy. In some embodiments, the patient has previously been treated with chemotherapy. In some embodiments, the patient has previously been treated targeted therapy.

In some embodiments, the patient has previously been treated with hormone therapy.

In some embodiments, the hormone therapy that the patient has previously been treated with is ovarian suppression. In certain embodiments, ovarian suppression is achieved by oophorectomy. In certain embodiments, ovarian suppression is achieved by administration of a GnRH antagonist.

In some embodiments, the hormone therapy that the patient has previously been treated with is an aromatase inhibitor. In some embodiments, the aromatase inhibitor is selected from exemestane (Aromasin®), letrozole (Femara®), and anastrozole (Arimidex®).

In some embodiments, the hormone therapy that the patient has previously been treated with is a selective ER modulator (SERM) other than lasofoxifene. In some embodiments, the selective ER modulator is selected from tamoxifen, raloxifene, bazedoxifene, toremifene, and ospemifene. In certain embodiments, the selective ER modulator is tamoxifen.

In some embodiments, the hormone therapy that the patient has previously been treated with is a selective ER degrader (SERD). In various embodiments, the selective ER degrader binds to the estrogen receptor and leads to the proteasomal degradation of the receptor. In some embodiments, the selective ER degrader is selected from fulvestrant, RAD1901, ARN-810 (GDC-0810), and AZD9496. In certain embodiments, the selective ER degrader is fulvestrant.

In some embodiments, the osteoporosis patient's breast cancer is in remission. In some of these embodiments, the patient's breast cancer is in partial remission. In some other of these embodiments, the patient's breast cancer is in complete remission.

### 4.1.4. Other Cancer Survivors with Osteoporosis

In some embodiments, the osteoporosis patient has previously been diagnosed with a malignancy other than breast cancer. In some of these embodiments, the osteoporosis patient has previously been diagnosed with ovarian cancer. In some other of these embodiments, the osteoporosis patient has previously been diagnosed with lung cancer.

In some embodiments, the osteoporosis patient has previously been diagnosed with estrogen receptor positive (ER+) malignancy other than breast cancer. In some other embodiments, the osteoporosis patient has previously been diagnosed with estrogen receptor negative (ER−) malignancy other than breast cancer. In various embodiments, the patient has been diagnosed with ER+ or ER− malignancy other than breast cancer by immunohistochemistry (IHC) performed on a sample of the patient's cancer.

In some embodiments, the patient's cancer has previously been treated with surgery. In some embodiments, the patient has previously been treated with radiation therapy. In some embodiments, the patient has previously been treated with chemotherapy. In some embodiments, the patient has previously been treated targeted therapy.

In some embodiments, the patient has previously been treated with hormone therapy.

In some embodiments, the hormone therapy that the patient has previously been treated with is ovarian suppression. In certain embodiments, ovarian suppression is achieved by oophorectomy. In certain embodiments, ovarian suppression is achieved by administration of a GnRH antagonist.

In some embodiments, the hormone therapy that the patient has previously been treated with is an aromatase inhibitor. In various embodiments, the aromatase inhibitor blocks the production of estrogen. In some embodiments, the aromatase inhibitor is selected from exemestane (Aromasin®), letrozole (Femara®), and anastrozole (Arimidex®).

In some embodiments, the hormone therapy that the patient has previously been treated with is a selective ER modulator (SERM) other than lasofoxifene. In some embodiments, the selective ER modulator is selected from tamoxifen, raloxifene, bazedoxifene, toremifene, and ospemifene. In certain embodiments, the selective ER modulator is tamoxifen.

In some embodiments, the hormone therapy that the patient has previously been treated with is a selective ER degrader (SERD). In various embodiments, the selective ER degrader binds to the estrogen receptor and leads to the proteasomal degradation of the receptor. In some embodiments, the selective ER degrader is selected from fulvestrant, RAD1901, ARN-810 (GDC-0810), and AZD9496. In certain embodiments, the selective ER degrader is fulvestrant.

In some embodiments, the osteoporosis patient's malignancy is in remission. In some of these embodiments, the patient's malignancy is in partial remission. In some other of these embodiments, the patient's malignancy is in complete remission.

### 4.2. Lasofoxifene

The selected patient is treated with an effective amount of lasofoxifene, a pharmaceutically acceptable salt thereof, or a prodrug thereof. In some preferred embodiments, lasofoxifene is administered to the selected patient as lasofoxifene tartrate.

The term “pharmaceutically acceptable salt” refers to non-toxic pharmaceutically acceptable salts. See Gould, International Journal of Pharmaceutics 33: 201-217 (1986) and Berge et al., Journal of Pharmaceutical Sciences 66(1): 1-19 (1977). Other salts well known to those in the art may, however, be used. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

Embodiments also include prodrugs of the compounds disclosed herein. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, H. Bundgaard, Elsevier, 1985.

Some of the crystalline forms for the compounds may exist as polymorphs and as such are intended to be included in the present invention. In addition, some of the compounds may form solvates with water (i.e., hydrates) or common organic solvents, and such solvates are intended to be encompassed by some embodiments.

Where the processes for the preparation of the compounds as disclosed herein give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers, cis-trans isomers, and enantiomers thereof are encompassed by some embodiments.

### 4.3. Pharmaceutical Compositions of the Invention

Methods for treatment of VVA and/or osteoporosis in cancer survivors, including breast cancer survivors, include administering a therapeutically effective amount of lasofoxifene, a pharmaceutically acceptable salt thereof, or a prodrug thereof. The lasofoxifene, the pharmaceutically acceptable salt, or the prodrug of the invention can be formulated in pharmaceutical compositions. In addition to lasofoxifene, a pharmaceutically acceptable salt thereof, or a prodrug thereof, the composition further comprises a pharmaceutically acceptable excipient, carrier, buffer, stabilizer or other materials well known to those skilled in the art. Such materials should be non-toxic and should not interfere with the efficacy of the active ingredient.

The precise nature of the carrier or other material can depend on the route of administration, e.g. oral, intravenous, transdermal, vaginal topical, or vaginal ring.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal oil, vegetable oil, mineral oil or synthetic oil. Physiological saline solution, dextrose or other saccharide solution or glycols such as ethylene glycol, propylene glycol or polyethylene glycol can also be included.

For parenteral administration, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Pharmaceutical compositions for vaginal topical administration can be in the form of ointment, cream, gel or lotion. The pharmaceutical compositions for vaginal topical administration often include water, alcohol, animal oil, vegetable oil, mineral oil or synthetic oil. Hydrocarbon (paraffin), wool fat, beeswax, macrogols, emulsifying wax or cetrimide can also be included.

Vaginal rings are typically constructed of biocompatible polymers or mixtures of polymers, such as polyolefins (e.g., polyethylene and polypropylene), polyurethanes, ethylene/vinylacetate copolymers, and silicone elastomers.

A composition can be administered alone or in combination with other treatments, either simultaneously or sequentially, dependent upon the condition to be treated.

### 4.4. Methods of Treatment

In another aspect, methods of administering an effective amount of lasofoxifene in the form of a pharmaceutical composition as described above for treatment of VVA and/or osteoporosis are provided.

The terms “treatment”, “treating”, and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or adverse effect, such as a symptom, attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

The term “effective amount” means a dose that produces the desired effect for which it is administered. The exact dose will depend on the purpose of the treatment, and will be ascertainable by one skilled in the art using known techniques. See Lloyd, The Art, Science and Technology of Pharmaceutical Compounding (1999).

### 4.4.1. Routes of Administration

In various embodiments, lasofoxifene is administered by oral, intravenous, transdermal, vaginal topical, or vaginal ring administration.

In some embodiments, lasofoxifene is administered to the patient by oral administration. In certain embodiments, lasofoxifene is administered at about 0.5 mg/day per os (p.o.) to about 10 mg/day per os. In some other embodiments, lasofoxifene is administered at more than 10 mg/day per os. In certain embodiments, lasofoxifene is administered once every day. In certain embodiments, lasofoxifene is administered once every two days. In certain embodiments, lasofoxifene is administered once every three days. In certain embodiments, lasofoxifene is administered once every four days. In certain embodiments, lasofoxifene is administered once every five days. In certain embodiments, lasofoxifene is administered once every six days. In certain embodiments, lasofoxifene is administered once every week. In certain embodiments, lasofoxifene is administered once every two weeks. In certain embodiments, lasofoxifene is administered once every three weeks. In certain embodiments, lasofoxifene is administered once every month.

In some embodiments, lasofoxifene is administered by vaginal topical administration. In certain embodiments, lasofoxifene is administered once every day. In certain embodiments, lasofoxifene is administered once every two days. In certain embodiments, lasofoxifene is administered once every three days. In certain embodiments, lasofoxifene is administered once every four days. In certain embodiments, lasofoxifene is administered once every five days. In certain embodiments, lasofoxifene is administered once every six days. In certain embodiments, lasofoxifene is administered once every week. In certain embodiments, lasofoxifene is administered once every two weeks. In certain embodiments, lasofoxifene is administered once every three weeks. In certain embodiments, lasofoxifene is administered once every month.

In some embodiments, lasofoxifene is administered to the patient by vaginal ring administration. In some of these embodiments, lasofoxifene is administered once every two weeks. In some of these embodiments, lasofoxifene is administered once every three weeks. In some of these embodiments, lasofoxifene is administered once every month. In some of these embodiments, lasofoxifene is administered once every two months. In some of these embodiments, lasofoxifene is administered once every three months. In some of these embodiments, lasofoxifene is administered once every four months.

In some embodiments, lasofoxifene is administered to the VVA or osteoporosis patient for one year. In some embodiments, lasofoxifene is administered to the patient for two years. In some embodiments, lasofoxifene is administered to the patient for three years. In some embodiments, lasofoxifene is administered to the patient for four years. In some embodiments, lasofoxifene is administered to the patient for five years. In some other embodiments, lasofoxifene is administered to the patient for more than five years. In certain embodiments, lasofoxifene is administered to the patient until the patient's disease progresses on therapy.

### 4.4.2. Combination Therapy

In various embodiments, lasofoxifene is administered either alone or in combination with other therapies. In certain embodiments, lasofoxifene is administered in combination with at least one other therapy. In some embodiments, lasofoxifene and other therapies are administered together (simultaneously). In some other embodiments, lasofoxifene and other therapies are administered at different times (sequentially).

In particular embodiments, the additional therapy that the patient is treated with is endocrine therapy. In various embodiments, the patient is treated with at least one line of additional endocrine therapy. In some embodiments, the patient is treated with one line of additional endocrine therapy. In some other embodiments, the patient is treated with multiple lines of additional endocrine therapy.

In some embodiments, the patient is treated with the additional endocrine therapy at the original doses. In some other embodiments, the patient is treated with the additional endocrine therapy at doses higher than original doses. In certain embodiments, the patient is treated with the additional endocrine therapy at doses lower than original doses.

In certain embodiments, the additional endocrine therapy is treatment with a selective ER modulator (SERM) other than lasofoxifene. In some of these embodiments, the selective ER modulator is selected from tamoxifen, raloxifene, bazedoxifene, toremifene, and ospermifene. In certain embodiments, the selective ER modulator is tamoxifen.

In certain embodiments, the additional endocrine therapy is treatment with a selective ER degrader (SERD). In some of these embodiments, the selective ER degrader is selected from fulvestrant, RAD1901, ARN-810 (GDC-0810), and AZD9496. In certain embodiments, the selective ER degrader is fulvestrant.

In certain embodiments, the additional endocrine therapy is treatment with an aromatase inhibitor. In some of these embodiments, the aromatase inhibitor is selected from exemestane (Aromasin®), letrozole (Femara®), and anastrozole (Arimidex®).

In various embodiments, the additional therapy is administration to the patient of an effective amount of a cell cycle inhibitor. In certain embodiments, the additional therapy is administration of an effective amount of cyclin-dependent kinase 4/6 (CDK4/6) inhibitor. In some embodiments, the additional therapy is a CDK4/6 inhibitor selected from the group of palbociclib, abemaciclib, and ribociclib.

In some embodiments, the additional therapy is an inhibitor of pathways that crosstalk with and activate the ER transcriptional activity. In various embodiments, the additional therapy a mammalian target of rapamycin (mTOR) inhibitor, phosphoinositide 3-kinase (PI3K) inhibitor, or heat shock protein 90 (HSP90) inhibitor.

In various embodiments, the additional therapy is the administration to the patient of an effective amount of a growth factor inhibitor. In certain embodiments, the additional therapy is a human epidermal growth factor receptor 2 (HER2) inhibitor. In some embodiments, the HER2 inhibitor is trastuzumab)(Herceptin®). In some other embodiments, the HER2 inhibitor is ado-trastuzumab emtansine (Kadcyla®).

In some embodiments, the additional therapy is the administering to the patient of an effective amount of a checkpoint inhibitor. In certain embodiments, the checkpoint inhibitor is an antibody. In some of these embodiments, the checkpoint inhibitor is an antibody specific for programmed cell death protein 1 (PD-1), programmed death-ligand 1 (PD-L1), or cytotoxic T-lymphocyte-associated protein 4 (CTLA-4). In some embodiments, the PD-1 antibody is pembrolizumab (Keytruda®) or nivolumab (Opdivo®). In some embodiments, the CTLA-4 antibody is ipilimumab (Yervoy®).

In certain embodiments, the additional therapy is administering to the patient an effective amount of cancer vaccine.

In certain embodiments, the additional therapy is administering to the patient an effective amount of bisphosphonate. In various embodiments, the bisphosphonate is selected from etidronate)(Didronel®), clodronate (Bonefos®, Loron®), tiludronate)(Skelid®), pamidronate (Aredia®), neridronate (Nerixia®), olpadronate, alendronate (Fosamax®), ibandronate (Boniva®), risedronate (Actonel®, Atelvia®), and zoledronate (Zometa®, Aclasta®).

In some embodiments, the additional therapy is administering to the patient an effective amount of receptor activator of nuclear factor kappa-B ligand (RANKL) inhibitor. In some of these embodiments, the inhibitor is denosumab (Prolia®, Xgeva®).

In certain embodiments, the additional therapy is administering to said patient an effective amount of calcitonin (Miacalcin®, Fortical®).

In certain embodiments, the additional therapy is administering to the patient an effective amount of an inhibitor of sclerostin.

In some embodiments, the additional therapy is administering to the patient an effective amount of a serotonin-norepinephrine reuptake inhibitor (SNRI), a selective serotonin reuptake inhibitor (SSRI), or gabapentin. In certain embodiments, the SNRI is venlafaxine (Effexor®).

### 4.4.3. Clinical Endpoints

### 4.4.3.1. Primary Clinical Endpoints

In various embodiments, the method comprises administering an amount of lasofoxifene effective to treat the symptoms of VVA and/or osteoporosis in cancer survivors.

In various embodiments, the method comprises administering an amount of lasofoxifene effective to treat the symptoms of vulvovaginal atrophy (VVA). In some embodiments, the method is effective to decrease vaginal pH, increase vaginal lubrication, and/or improve vaginal cell maturation index. In some embodiments, the method is effective to decrease vaginal pH, increase vaginal lubrication, and/or improve vaginal cell maturation index in women who are concurrently being treated with one or more drugs causing or predisposing to VVA.

In some embodiments, the method reduces one or more symptoms of sexual dysfunction. In some embodiments, the method reduces one or more symptoms of sexual dysfunction in women who are concurrently being treated with one or more drugs causing or predisposing to sexual dysfunction.

In some embodiments, the method treats hot flashes. In some embodiments, the method treats hot flashes in women who are concurrently being treated with one or more drugs causing or predisposing to hot flashes.

In various embodiments, the method comprises administering an amount of lasofoxifene effective to treat the symptoms of osteoporosis. In some embodiments, the method is effective to prevent fracture and/or bone loss. In some embodiments, the method is effective to prevent fracture and/or bone loss in women who are concurrently being treated with one or more drugs causing or predisposing to osteoporosis.

### 4.4.3.2. Secondary Clinical Endpoints

In some embodiments, the method comprises administering an amount of lasofoxifene effective to increase disease-free survival, reduce recurrence, increase time to recurrence, reduce metastasis, and/or increase duration of progression-free survival in women who have previously been diagnosed with cancer.

In certain embodiments, the method comprises administering an amount of lasofoxifene effective to increase disease-free survival, reduce recurrence, increase time to recurrence, reduce metastasis, and/or increase duration of progression-free survival in women who have previously been diagnosed with breast cancer.

In some of these embodiments, the method comprises administering an amount of lasofoxifene effective to increase disease-free survival, reduce recurrence, increase time to recurrence, reduce metastasis, and/or increase duration of progression-free survival in women who have previously been diagnosed with ER+ breast cancer.

In some other of these embodiments, the method comprises administering an amount of lasofoxifene effective to increase disease-free survival, reduce recurrence, increase time to recurrence, reduce metastasis, and/or increase duration of progression-free survival in women who have previously been diagnosed with ER− breast cancer.

In some embodiments, the method comprises administering an amount of lasofoxifene effective to increase one or more quality of life measures selected from joint ache, urogenital symptoms, bone loss, and bone fractures.

### 5. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1-114. (canceled)

115. A method of treating vulvovaginal atrophy (VVA) in women who have previously been diagnosed with breast cancer, comprising:
a) selecting for treatment a patient with VVA who has previously been diagnosed with either i) estrogen receptor positive (ER+) breast cancer or ii) estrogen receptor negative (ER−) breast cancer and who has previously been treated with an aromatase inhibitor, a selective ER modulator (SERM) other than lasofoxifene, or a selective ER degrader (SERD); and
b) administering to the selected patient lasofoxifene or a pharmaceutically acceptable salt thereof, in an amount effective to treat VVA.

116. The method of claim 115, wherein lasofoxifene is administered as lasofoxifene tartrate.

117. The method of claim 115, wherein lasofoxifene is administered by oral, intravenous, transdermal, vaginal topical, or vaginal ring administration.

118. The method of claim 117, wherein lasofoxifene is administered by oral administration.

119. The method of claim 118, wherein lasofoxifene is administered at about 0.5 mg/day per os to about 10 mg/day per os.

120. The method of claim 117, wherein lasofoxifene is administered by vaginal topical administration.

121. The method of claim 117, wherein lasofoxifene is administered by vaginal ring administration.

122. The method of claim 115, wherein lasofoxifene is administered once every day, once every two days, once every three days, once every four days, once every five days, once every six days, once every week, once every two weeks, once every three weeks, or once every month.

123. The method of claim 115, further comprising treating said patient with at least one additional endocrine therapy.

124. The method of claim 123, wherein the additional endocrine therapy is treatment with a second selective ER modulator (SERM).

125. The method of claim 123, wherein the additional endocrine therapy is treatment with a selective ER degrader (SERD).

126. The method of claim 123, wherein the additional endocrine therapy is treatment with an aromatase inhibitor.

127. The method of claim 115, further comprising administering to said patient an effective amount of cyclin-dependent kinase 4/6 (CDK4/6) inhibitor.

128. The method of claim 127, wherein said CDK4/6 inhibitor is palbociclib, abemaciclib, or ribociclib.

129. The method of claim 115, further comprising administering to said patient an effective amount of mammalian target of rapamycin (mTOR) inhibitor, phosphoinositide 3-kinase (PI3K) inhibitor, or heat shock protein 90 (HSP90) inhibitor.

130. The method of claim 115, further comprising administering to said patient an effective amount of human epidermal growth factor receptor 2 (HER2) inhibitor.

131. The method of claim 130, wherein said HER2 inhibitor is trastuzumab (Herceptin®) or ado-trastuzumab emtansine (Kadcyla®).

132. The method of claim 115, further comprising administering to said patient an effective amount of a checkpoint inhibitor.

133. The method of claim 132, wherein said checkpoint inhibitor is an antibody specific for programmed cell death protein 1 (PD-1), programmed death-ligand 1 (PD-L1), or cytotoxic T-lymphocyte-associated protein 4 (CTLA-4).

134. The method of claim 133, wherein said PD-1 antibody is pembrolizumab (Keytruda®) or nivolumab (Opdivo®).

135. The method of claim 133, wherein said CTLA-4 antibody is ipilimumab (Yervoy®).

136. The method of claim 115, further comprising administering to said patient an effective amount of cancer vaccine.

137. The method of claim 115, comprising administering an amount of lasofoxifene sufficient to decrease vaginal pH, increase vaginal lubrication, and/or improve vaginal cell maturation index in women who are concurrently being treated with one or more drugs causing or predisposing to VVA.

138. The method of claim 115, comprising administering an amount of lasofoxifene sufficient to reduce one or more symptoms of sexual dysfunction in women who are concurrently being treated with one or more drugs causing or predisposing to sexual dysfunction.

139. The method of claim 115, comprising administering an amount of lasofoxifene sufficient to reduce one or more symptoms of hot flashes in women who are concurrently being treated with one or more drugs causing or predisposing to hot flashes.

140. The method of claim 115, comprising administering an amount of lasofoxifene sufficient to reduce recurrence of breast cancer, increase time to recurrence of breast cancer, reduce metastasis of breast cancer to bone, and/or increase duration of breast cancer progression-free survival.

141. The method of claim 115, wherein lasofoxifene or salt thereof is administered in an amount sufficient to increase one or more quality of life measures selected from: joint ache, urogenital symptoms, bone loss, and bone fractures.

142. The method of claim 115, wherein the patient's breast cancer is in remission.
